(19) United States
(12) Patent Application Publication
(10) Pub. No.: US 2023/0090660 A1
(43) Pub. Date: Mar. 23, 2023

(54) BENEFICIAL EFFECT OF POACEAE AND/OR LEGUMINOSEAE FIBER ON SURVIVABILITY AND METABOLIC ACTIVITY OF BIFIDOBACTERIUM LONGUM

(71) Applicant: SOCIETE DES PRODUITS NESTLE S.A., Vevey (CH)

(72) Inventors: STEPHANE DUBOUX, St-Prex (CH); LISA MARCELA LAMOTHE, Lausanne 26 (CH); ROSA ARAGAO BORNER, Epalinges (CH); FLORENCE ROCHAT, Montreux (CH)

(21) Appl. No.: 17/999,077

(22) PCT Filed: May 18, 2021
(86) PCT No.: PCT/EP2021/063165

§ 371 Date: Nov. 17, 2022

(30) Foreign Application Priority Data
May 20, 2020 (EP) 20175627.7

(51) Int. Cl. A23L 33/22

(52) U.S. Cl. CPC A23L 33/22

(57) ABSTRACT

The present invention relates to the use of a Poaceae and/or a Leguminoseae plant fiber to improve survival of a Bifidobacterium probiotic, wherein said plant fiber has an insoluble fraction of between 40 to 80% (w/w).

Probiotics, often bacteria, are live microorganisms which provide a health benefit to the host. It is commonly accepted that those bacteria should resist the gastric passage and be alive at the targeted site of action in the gut to confer said health benefit. Unfortunately, some of those probiotic bacteria have a relatively poor resistance to gastro-intestinal conditions and therefore, only a small portion of the bacteria consumed survives. Some prebiotic ingredients have been postulated to improve the efficacy of probiotics in combinations described as synbiotics, however the evidence for this is inconsistent. Also, such prebiotic ingredients are not perceived as “natural” by consumers because they tend to be synthesized from monosaccharides or extracted and purified from their natural sources.

### SUMMARY OF THE INVENTION

It has been surprisingly demonstrated that, compared to soluble fibers, certain insoluble fibers improve the survival of Bifidobacterium during conditions which mimic gastro-intestinal tract passage in a subject.

The superior effect has been demonstrated for Bifidobacterium longum NCC 3001 (ATCC BAA-999, hereafter B. longum NCC 3001), particularly when it is combined with a Leguminoseae plant fiber (e.g. pea cell wall fiber) or a Poaceae fiber (e.g. corn fiber).

Without wishing to be bound by theory, it is believed that pea cell wall fiber and corn fiber are optimal substrates for B. longum 999 and may serve as a “shield” against harsh gastro-intestinal conditions. B. longum 999 is able to reach the large intestine in greater counts with improved viability, and reside there for a longer time.

In a first aspect, the invention provides the use of a Poaceae and/or Leguminoseae plant fiber to improve survival of a Bifidobacterium probiotic, wherein said plant fiber has an insoluble fraction of between 40 to 80% (w/w).

In a second aspect, the invention provides a composition comprising an effective amount of a Poaceae and/or Leguminoseae plant fiber and a Bifidobacterium probiotic, wherein said plant fiber has an insoluble fraction of between 40 to 80% (w/w).

In a third aspect, the invention provides a composition comprising an effective amount of a Poaceae and/or Leguminoseae plant fiber and a Bifidobacterium probiotic, wherein said plant fiber has an insoluble fraction of between 40 to 80% (w/w), and wherein said Bifidobacterium probiotic is obtained by a process comprising the steps of

a. Fermenting the Bifidobacterium in a bacterial growth medium; and

b. Harvesting the cultured Bifidobacterium probiotic.

In a fourth aspect, the invention provides a composition as described herein for use as a medical food.

### BRIEF DESCRIPTION OF THE FIGURES

FIG. 1: Schematic of the adapted SHIME system containing a reactor that consecutively simulates stomach and small intestinal conditions under both fed and fasted conditions.

FIG. 2: pH profile of the incubation mimicking upper GIT passage conditions. Samples were taken at the start of the stomach incubation (ST0), the end of the stomach incubation (ST2), the start of the small intestinal incubation (SI0), the start of the absorption phase in the small intestinal incubation (SI0.4) and the end of the small intestinal incubation (SI4).

FIG. 3: Survival of NCC 3001 in presence of different insoluble & soluble fibers in a microsystem mimicking GI transit.

FIG. 4: Improvement of B. longum survival by corn fiber & pea fiber in a complex in vitro model (SHIME®); measured through Colony Forming Units (CFU).

FIG. 5: Improvement of metabolic activity of B. longum by corn fiber and pea fiber in a complex in vitro model (SHIME®); measured by esterase activity using flow cytometry.

### EMBODIMENTS OF THE INVENTION

The present invention relates in general to the use of a Poaceae and/or a Leguminoseae plant fiber to improve survival of a Bifidobacterium probiotic, wherein said plant fiber has an insoluble fraction of between 40 to 80% (w/w).

Poaceae fibers such as corn fiber with high insoluble fractions are particularly useful for improving survival. In some embodiments, the Poaceae fiber has an insoluble fraction of between 70 to 80% (w/w). In some embodiments, the Poaceae fiber comprises corn fiber. In some embodiments, the corn fiber is a corn fiber mix comprising between 30 to 60% (w/w) corn fiber, or about 45% (w/w) corn fiber.

Leguminoseae fibers such as pea cell wall fiber with low insoluble fractions are also useful for improving survival. In some embodiments, the Leguminoseae fiber has an insoluble fraction of between 40-50% (w/w) of total plant fiber. In some embodiments, the Leguminoseae fiber is pea cell wall fiber.

Poaceae and Leguminoseae plant fibers can be used to improve the survival of a Bifidobaterium probiotic. In some embodiments, the probiotic belongs to the Bifidobacterium longum species. In some embodiments, the probiotic is B. longum subsp. longum. In some embodiments, the probiotic is B. longum NCC 3001 (ATCC BAA-999).

In some embodiments, the probiotic is B. longum NCC 3001 (ATCC BAA-999) and the plant fiber is corn fiber.

In one embodiment, B. longum NCC 3001 (ATCC BAA-999) is pre-conditioned by growing in the presence of corn fiber.

In some embodiments, the probiotic is B. longum NCC 3001 (ATCC BAA-999) and the plant fiber is pea fiber.

In one embodiment, B. longum NCC 3001 (ATCC BAA-999) is pre-conditioned by growing in the presence of corn fiber.

In one embodiment, the improved survival is improved gastric survival. In one embodiment, the improved survival is improved duodenal survival.

In one embodiment, survival is improved compared to when grown in the absence of fiber. In one embodiment, survival is improved compared to when grown in the presence of inulin. In one embodiment, survival is improved compared to when grown in the presence of resistant dextrin.

Poaceae and Leguminoseae plant fibers can be used to improve attributes of Bifidobaterium. In some embodiments, these attributes include probiotic metabolic activity, metabolite production, and/or bifidogenic effect. In one embodiment, the attribute is probiotic metabolic activity of a Bifidobacterium probiotic during gastro-intestinal passage in a subject. In one embodiment, the attribute is metabolite production of a Bifidobacterium probiotic during gastro-intestinal passage in a subject.

In one embodiment, the attribute is bifidogenic effect of a Bifidobacterium probiotic during gastro-intestinal passage in a subject. In one embodiment, the attribute is improved compared to when grown in the absence of fiber. In one embodiment, the attribute is improved compared to when grown in the presence of inulin. In one embodiment, the attribute is improved compared to when grown in the presence of resistant dextrin. Metabolic activity can be measured, for example by measuring esterase activity.

The present invention further relates to a composition comprising an effective amount of a Poaceae and/or Leguminoseae plant fiber and Bifidobacterium probiotic, wherein said plant fiber has an insoluble fraction of between 40-80% (w/w).

Growing the Bifidobacterium in presence of the prebiotic ingredients appears to have a beneficial effect, particularly for Poaceae plant fibers such as corn fiber.

In one embodiment, the composition comprises an effective amount of a Poaceae and/or Leguminoseae plant fiber and Bifidobacterium probiotic, wherein said plant fiber has an insoluble fraction of between 40-80% (w/w), and wherein said Bifidobacterium probiotic is obtained by a process comprising the steps of

a. Fermenting the Bifidobacterium in a bacterial growth medium; and

b. Harvesting the cultured Bifidobacterium probiotic.

In some embodiments, the bacterial growth medium comprises a Poaceae plant fiber, for example corn fiber.

In one embodiment, the composition is a food, a medical food, a tube feed, or a nutritional supplement.

In one embodiment, the food is selected from milk, yoghurt, curd, cheese, fermented milks, milk based fermented products, rice based products, milk based powders, infant formulae and pet food.

In one embodiment, the composition is a pharmaceutical composition wherein the pharmaceutical composition comprises one or more pharmaceutically acceptable carriers, diluents and/or excipients.

Definitions

As used in this disclosure and the appended claims, the singular forms “a,” “an” and “the” include plural referents unless the context clearly dictates otherwise. Thus, for example, reference to “a bacterial strain” or “the bacterial strain” includes two or more bacterial strains.

The words “comprise,” “comprises” and “comprising” are to be interpreted inclusively rather than exclusively. Likewise, the terms “include,” “including” and “or” should all be construed to be inclusive, unless such a construction is clearly prohibited from the context.

Nevertheless, the compositions disclosed herein may lack any element that is not specifically disclosed. Thus, a disclosure of an embodiment using the term “comprising” includes a disclosure of embodiments “consisting essentially of and “consisting of” the components identified. Similarly, the methods disclosed herein may lack any step that is not specifically disclosed herein. Thus, a disclosure of an embodiment using the term “comprising” includes a disclosure of embodiments “consisting essentially of and “consisting of the steps identified.

The term “and/or” used in the context of “X and/or Y” should be interpreted as “X,” or “Y,” or “X and Y.” Where used herein, the terms “example” and “such as,” particularly when followed by a listing of terms, are merely exemplary and illustrative and should not be deemed to be exclusive or comprehensive. Any embodiment disclosed herein can be combined with any other embodiment disclosed herein unless explicitly stated otherwise.

As used herein, “about” and “approximately” are understood to refer to numbers in a range of numerals, for example the range of −10% to +10% of the referenced number, preferably within −5% to +5% of the referenced number, more preferably within −1% to +1% of the referenced number, most preferably within −0.1% to +0.1% of the referenced number.

Furthermore, all numerical ranges herein should be understood to include all integers, whole or fractions, within the range. The term “between” includes the end points of the identified range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

However, while the term “subject” is often used herein to refer to a human, the present disclosure is not so limited. Accordingly, the terms “individual” and “patient” refer to any animal, mammal that can benefit from the treatment.

As used herein, an “effective amount” is an amount that prevents a deficiency, treats a disorder, condition, or disease in a subject or, more generally, reduces symptoms, manages progression of the diseases or provides a nutritional, physiological, or medical benefit to the subject.

The terms “treatment” and “treating” include any effect that results in the improvement of the condition or disorder, for example lessening/ameliorating, reducing, modulating, or eliminating the condition or disorder. The term does not necessarily imply that a subject is treated until total recovery. Non-limiting examples of “treating” or “treatment of a condition or disorder include: (1) inhibiting the condition or disorder, i.e. arresting the development of the condition or disorder or its clinical symptoms and (2) relieving the condition or disorder, i.e. causing the temporary or permanent regression of the condition or disorder or its clinical symptoms. A treatment can be patient- or doctor-related.

The terms “prevention” or “preventing” mean causing the clinical symptoms of the referenced condition or disorder to not develop or reducing the risk of their development in an individual. The individual may be exposed or predisposed to the condition or disorder but does not yet experience or display symptoms of the condition or disorder. The terms “condition” and “disorder” mean any disease, condition, symptom, or indication.

The relative term “optimize or optimise” as used herein mean to improve, increase, or enhance.

The terms “food,” “food product” and “food composition” mean a product or composition that is intended for ingestion by an individual such as a human and provides at least one nutrient to the individual. The food product may be, for example, a nutritionally complete formula (for example an infant formula or a clinical nutrition product), a dairy product, a beverage powder, a dehydrated soup, a dietary supplement, a meal replacement, a nutritional bar, a cereal, a confectionery product or a complete and balanced pet food, e.g. dry pet food composition or wet pet food composition.

The term “pet food” or “pet food composition” means any food composition intended to be consumed by a pet. The term “pet” means any animal which could benefit from or enjoy the compositions provided by the present disclosure. For example, the pet can be an avian, bovine, canine, equine, feline, hircine, lupine, murine, ovine, or porcine animal, but the pet can be any suitable animal. In one aspect, the pet can be a companion animal. As such, the term “cat food composition” means any food composition intended to be ingested by a cat and the term “dog food composition” means any composition intended to be ingested by a dog.

The term “complete and balanced” when referring to a food composition means a food composition that contains all known required nutrients in appropriate amounts and proportions based on recommendations of recognized authorities in the field of animal nutrition, and are therefore capable of serving as a sole source of dietary intake to maintain life or promote production, without the addition of supplemental nutritional sources. Nutritionally balanced pet food and animal food compositions are widely known and widely used in the art, e.g., complete and balanced food compositions formulated according to standards established by the Association of American Feed Control Officials (AAFCO).

The term “companion animal” means a dog or a cat.

“Wet pet food” means a pet food having a moisture content from about 50% to about 90%, and in one aspect, from about 70% to about 90%. “Dry pet food” means a pet food having a moisture content less than about 20%, and in one aspect, less than about 15%, and in a specific aspect, less than about 10%. “Semi-moist food” means a pet food having a moisture content from about 20% to about 50%, and in one aspect, from about 25% to about 35%.

In some embodiments, the pet food compositions can comprise a protein. The protein can be crude protein material and may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include beef, pork, lamb, equine, poultry, fish, and mixtures thereof. In one embodiment, the food compositions can comprises the protein in amounts from about 5%, 10%, 15%, 20%, 25%, 30%, 35%, 40%, 45%, 50%, 55%, or even 60% to about 25%, 30%, 35%, 40%, 45%, 50%, 55%, 60%, 65%, or even 70%, including various subranges within these amounts. In one aspect, the protein can be from about 30% to about 55% of the food composition.

In some embodiments, the pet food compositions can comprise carbohydrates. Generally, any type of carbohydrate can be used in the food compositions. Examples of suitable carbohydrates include grains or cereals such as rice, corn, millet, sorghum, alfalfa, barley, soybeans, canola, oats, wheat, rye, triticale and mixtures thereof. The compositions may also optionally comprise other materials such as dried whey and other dairy by-products. In one embodiment, the carbohydrate comprises from about 5% to about 10% of the food composition. In another embodiment, the carbohydrate comprises from about 10% to about 15% of the food compositions. In other aspects, the carbohydrate can be present in amounts from about 5%, 10%, 15%, 20%, 25%, 30%, 35%, or even 40% to about 10%, 15%, 20%, 25%, 30%, 35%, 40%, 45%, or even 50%.

In some embodiments, the pet food compositions can include fat. Examples of suitable fats include animal fats and vegetable fats. In one aspect, the fat source can be an animal fat source such as tallow or poultry fat. Vegetable oils such as corn oil, sunflower oil, safflower oil, grape seed oil, soy bean oil, olive oil and other oils rich in monounsaturated and polyunsaturated fatty acids, may also be used. In one embodiment, the food compositions can comprises the fat in amounts from about 5%, 10%, 15%, 20%, 25%, 30%, or even 35% to about 10%, 15%, 20%, 25%, 30%, 35%, or even 40%, including various subranges within these amounts. In one aspect, the fat comprises from about 25% to about 35% of the food composition.

The administration of the pet food composition can be performed on as-needed basis, an as-desired basis, a regular basis, or intermittent basis. In one aspect, the pet food composition can be administered to the animal on a regular basis. In one aspect, at least weekly administration can be performed. More frequent administration or consumption, such as twice or three times weekly, can be performed in certain embodiments. In one aspect, an administration regimen can comprise at least once daily consumption.

Generally, the pet food composition can be suitable for consumption by an animal, including companion animals such as dogs and cats, as a meal, component of a meal, a snack, supplement, or a treat. Such compositions can include complete foods intended to supply the necessary dietary requirements for an animal. Examples of such food compositions include but are not limited to dry foods, wet foods, semi-moist foods, drinks, bars, frozen prepared foods, shelf prepared foods, and refrigerated prepared foods.

As discussed herein, the pet food compositions may be administered to an animal alone as a complete nutritionally balanced diet, as a supplement, or in conjunction with dietary supplements, vitamins and/or other nutritionally beneficial agents familiar to one of skill in the art, as part of an overall wellness program for the animal. Compositions of the invention may also be useful as a veterinary therapeutic product.

As such, the compositions may optionally contain a carrier, diluent, or an excipient, the suitability of which for the intended use being familiar to one of skill in the art.

The term “prebiotic” means a substrate that is selectively utilized by host microorganisms conferring a health benefit' (Expert consensus document: The International Scientific Association for Probiotics and Prebiotics (ISAPP) consensus statement on the definition and scope of prebiotics, Nature Reviews Gastroenterology & Hepatology, 2017, 14, 491-502).

The term “probiotic” means live microorganisms that, when administered in adequate amounts, confer a health benefit on the host (FAO/WHO, 2002). The microbial cells are generally bacteria or yeasts.

The term “synbiotic” means nutritional compositions or food supplements combining both probiotic(s) and prebiotic(s) and in which the prebiotic(s) selectively favours the probiotic(s) (see DeVrese and Schrezenmeir, Probiotics, prebiotics and synbiotics in food biotechnology, Springer Berlin Heidelberg, pp 1-66. The term “synbiotic effect” refers herein to the increase of the advantageous health effect of the synbiotic compared to the effect of the probiotic alone.

The compositions of the present disclosure, including the many embodiments described herein, can comprise, consist of, or consist essentially of the essential elements and limitations described herein, as well as any additional or optional ingredients, components, or limitations described herein or otherwise useful in a diet.

### DETAILED DESCRIPTION

Use of Plant Fiber

The invention relates in general to the use of a Poaceae and/or a Leguminoseae plant fiber to improve survival of a Bifidobacterium probiotic, for example during gastro-intestinal tract passage in a subject.

In particular, the invention relates to the use of Poaceae and/or a Leguminoseae plant fiber to improve survival of a Bifidobacterium probiotic during gastro-intestinal tract passage in a subject, wherein the plant fiber has an insoluble fraction of between 40 to 80% (w/w).

The inventors surprisingly demonstrated that soluble fibers such as resistant dextrin or inulin did not support the survival of the probiotic in conditions that mimic gastric passage and duodenal passage, while non-soluble fibers such as pea fiber & corn fiber did support survival of the probiotic.

Accordingly, the invention also relates to use of Poaceae fiber, for example corn fiber, to improve survival of B. longum during gastric passage, or to improve survival of B. longum during duodenal passage, for example when compared to use of resistant dextrin or inulin.

The invention also relates to use of Leguminoseae fiber, for example pea cell wall fiber, to improve survival of B. longum during gastric passage, or to improve survival of B. longum during duodenal passage, for example when compared to use of resistant dextrin or inulin.

Survivability of the probiotic through upper GIT passage surprisingly improved when administered together with either corn fiber or pea fiber.

Accordingly, the invention also relates to use of Leguminoseae fiber, for example pea cell wall fiber, to improve survival of B. longum in the small intestine, for example when compared to absence of pea cell wall fiber, or to use of Poaceae fiber, for example corn fiber, to improve survival of B. longum in the small intestine, for example when compared to absence of corn fiber.

The number of metabolically active B. longum NCC3001 cells was surprisingly improved when administered together with pea fiber or corn fiber. In conditions that mimic the stomach, the best performance was seen using the synbiotic mix of B. longum NCC 3001 with corn fiber. The probiotic was grown with the fiber prior to administration to the upper GIT incubations. The best effect was seen when the fiber was pre-conditioned.

Accordingly, the invention also relates to use of Leguminoseae fiber, for example pea cell wall fiber, to improve metabolic activity of B. longum in the colon and upon stomach passage, for example when compared to absence of pea cell wall fiber, or to use of Poaceae fiber, for example corn fiber, to improve metabolic activity of B. longum in the stomach, for example when compared to absence of corn fiber.

In particular, the invention relates to use of Poaceae fiber, for example corn fiber, to improve metabolic activity of B. longum in the colon and upon stomach passage, for example when compared to absence of corn fiber, wherein the Poaceae fiber is grown in the presence of B. longum before administration to a subject.

The use of Poaceae and/or a Leguminoseae plant fiber improves survival of a Bifidobacterium compared to soluble fiber, for example compared to inulin or dextran.

Method of Improving Survival

The invention further relates to a method of improving survival of Bifidobacterium longum wherein said method comprises growing Bifidobacterium longum in a culture medium, characterised in that said culture medium comprises a Poaceae and/or Leguminoseae plant fiber or a combination thereof. Preferably the Bifidobacterium longum is Bifidobacterium longum subsp. longum.

In one embodiment, the improved survival is improved gastric survival. In one embodiment, the improved survival is improved duodenal survival.

In one embodiment, survival is improved compared to when grown in the absence of fiber. In one embodiment, survival is improved compared to when grown in the presence of inulin. In one embodiment, survival is improved compared to when grown in the presence of resistant dextrin.

Composition

The invention further relates to a composition comprising an effective amount of a plant fiber and Bifidobacterium probiotic, wherein said plant fiber has an insoluble fraction of between 40-80% (w/w).

In particular, the invention relates to a composition comprising an effective amount of a Poaceae and/or Leguminoseae plant fiber and Bifidobacterium probiotic, wherein said plant fiber has an insoluble fraction of between 40-80% (w/w).

In particular, the invention relates to a composition comprising an effective amount of a Poaceae and/or Leguminoseae plant fiber and Bifidobacterium probiotic, wherein said plant fiber has an insoluble fraction of between 40-80% (w/w), and wherein said Bifidobacterium probiotic is obtained by a process comprising the steps of
a. Fermenting the Bifidobacterium probiotic in a bacterial growth medium; and
b. Harvesting the cultured Bifidobacterium probiotic.

In one embodiment, the bacterial growth medium comprises Poaceae and/or Leguminoseae plant fiber.

The composition of the present invention may be in the form of a food, a medical food, a tube feed, a nutritional composition, or a nutritional supplement. The term “nutritional supplement” refers to a product which is intended to supplement the general diet of a subject.

In one embodiment, the food is selected from milk, yoghurt, curd, cheese, fermented milks, milk based fermented products, rice based products, milk based powders, infant formulae and pet food.

The composition may be in the form of a medical food. The term “medical food” as used herein refers to a food product specifically formulated for the dietary management of a medical disease or condition. The medical food may be administered under medical supervision. The medical food may be for oral ingestion or tube feeding.

The composition may be in the form of a tube feed. The term “tube feed” refers to a product which is intended for introducing nutrients directly into the gastrointestinal tract of a subject by a feeding tube. A tube feed may be administered by, for example, a feeding tube placed through the nose of a subject (such as nasogastric, nasoduodenal, and nasojejunal tubes), or a feeding tube placed directly into the abdomen of a subject (such as gastrostomy, gastrojejunostomy, or jejunostomy feeding tube).

The composition may in the form of a pharmaceutical composition and may comprise one or more suitable pharmaceutically acceptable carriers, diluents and/or excipients.

Examples of such suitable excipients for compositions described herein may be found in the “Handbook of Pharmaceutical Excipients”, 2nd Edition, (1994), Edited by A Wade and P J Weller.

Acceptable carriers or diluents for therapeutic use are well known in the pharmaceutical art, and are described, for example, in “Remington's Pharmaceutical Sciences”, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s) and/or solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like.

Preservatives, stabilisers, dyes and even flavouring agents may be provided in the composition. Examples of preservatives include sodium benzoate, sorbic acid and esters of p hydroxybenzoic acid. Antioxidants and suspending agents may be also used.

Nutritionally acceptable carriers, diluents and excipients include those suitable for human or animal consumption that are used as standard in the food industry. Typical nutritionally acceptable carriers, diluents and excipients will be familiar to the skilled person in the art.

The composition may be in the form of a tablet, dragee, lozenges, capsule, gel cap, powder, granule, solution, emulsion, suspension, coated particle, spray-dried particle or pill.

It is clear to those skilled in the art that an ideal dose will depend on the subject to be treated, its health condition, sex, age, or weight, for example, and the route of administration. The dose to be ideally used will consequently vary but can be determined easily by those of skill in the art.

However, generally, it is preferred if the composition of the present invention comprises between 10⁶ and 10¹⁰ cfu and/or between 10⁶ and 10¹⁰ cells of Bifidobacterium longum subsp longum per daily dose. It may also comprise between 10⁶ and 10¹¹ cfu and/or between 10⁶ and 10¹¹ cells of Bifidobacterium longum subsp longum per g of the dry weight of the composition.

Poaceae Plant Fiber

Poaceae plant fiber may be derived from Corn, Barley, Oats, Rice, Rye, Sorghum, Wheat, or Millet. Preferably, poaceae plant fiber may be derived from Corn, Barley, Oats, Rice, Rye, Sorghum, or Millet. Preferably, the plant fiber is derived from Corn.

The plant fiber may be produced as a byproduct of milling, for example in the form of a fiber mix.

When the plant fiber is a Poaceae fiber, the insoluble fraction is preferably between 70-80% (w/w).

The corn fiber may be in the form of a corn fiber mix. The mix may comprise between 30 to 60% (w/w), or 35 to 55% (w/w), or 40 to 50% (w/w), or about 45% (w/w) dietary fiber; and/or between 20 to 50% (w/w), or 30 to 40% (w/w), or about 33% (w/w) corn bran; and/or between 1 to 10% (w/w) wheat flour, preferably whole grain wheat flour, preferably about 5.3% (w/w) whole grain wheat flour; and/or between 1 to 10% (w/w) dextrin, preferably resistant dextrin, preferably about 5% resistant dextrin; and/or between 1 to 5% (w/w) gum, preferably guar gum, preferably about 0.8% guar gum; and/or between 1 to 5% (w/w) carboxymethylcellulose, preferably about 0.5% (w/w) carboxymethylcellulose.

In some embodiments, the Poaceae fiber comprises corn fiber, preferably about 45% (w/w) corn fiber. In some embodiments, said Poaceae fiber further comprises about 5% (w/w) resistant dextrin, about 2.5% (w/w) wheat bran, about 1% (w/w) guar gum and about 0.5% (w/w) carboxymethylcellulose.

The Bifidobacterium probiotic may be pre-conditioned with the Poaceae plant fiber. For example, B. longum NCC 3001 may be pre-conditioned by growing in the presence of corn fiber.

Leguminoseae Plant Fiber

The plant fiber may be a Leguminoseae fiber. When the plant fiber is a Leguminoseae fiber, the insoluble fraction is preferably between 40-50% (w/w). Leguminoseae include a number of important agricultural and food plants, including Pisum sativum (pea), Glycine max (soybean), Phaseolus (beans), Cicer arietinum (chickpeas), Medicago sativa (alfalfa), Arachis hypogaea (peanut), Ceratonia siliqua (carob), and Glycyrrhiza glabra (liquorice). Preferably, the Leguminoseae fiber is selected from Pisum sativum (pea), Phaseolus (beans), Cicer arietinum (chickpeas), Arachis hypogaea (peanut), Ceratonia siliqua (carob), and Glycyrrhiza glabra (liquorice). Preferably, the Leguminoseae fiber is selected from Pisum sativum (pea), and Cicer arietinum (chickpeas). Preferably, the Leguminoseae fiber is pea fiber. Preferably the pea fiber is pea cell wall fiber.

The Bifidobacterium probiotic may be pre-conditioned with the Leguminoseae plant fiber. For example, B. longum NCC3001 may be pre-conditioned by growing in the presence of pea cell wall fiber.

Bifidobacterium Probiotic

The Bifidobacterium probiotic of the invention may be Bifidobacterium longum, Bifidobacterium animalis ssp. lactis, or Bifidobacterium breve. Most preferably, it is Bifidobacterium longum, for example B. longum subsp. longum, B. longum subsp. infantis, or B. longum subsp. suis, preferably B. longum subsp. longum. The B. longum subsp. longum can be selected from Bifidobacterium longum ATCC BAA-999, Bifidobacterium longum ATCC 15707, and Bifidobacterium longum CNCM 1-2618. Most preferably, it is Bifidobacterium longum ATCC BAA-999 (NC3001).

B. longum ATCC BAA-999 is also known as NCC3001 and may be obtained commercially from specialist suppliers, for example from Morinaga Milk Industry Co. Ltd. of Japan under the trademark BB536. The term “B. longum ATCC BAA-999” includes the bacterium, parts of the bacterium, and/or a growth medium fermented by the bacterium.

B. longum ATCC BAA-999 was deposited and is publicly available in ATCC's general bacteriology collection by Tomoko Yaeshima, Morinaga Milk Industry Co. Ltd, Higashihara, Zama-City, Kanagawa-pref., Japan, on Jun. 7, 2004 at AMERICAN TYPE CULTURE COLLECTION, 10801 University Boulevard, Manassas, Va. 20110-2209.

The B. longum ATCC BAA-999 may be cultured according to any suitable method. B. longum ATCC BAA-999 may be added to a composition in any technically feasible form e.g. a freeze-dried or spray-dried form.

Strain ATCC 15707 was deposited prior to 1990 and is commercially available. Strain CNCM I-2618 was deposited by Nestec S.A., avenue Nestlé 55, 1800 Vevey, Switzerland on Jan. 29, 2001. Since then, Nestec S.A. has merged into Société des Produits Nestlé S.A. Accordingly, Societe des Produits Nestlé S.A. is the successor in title of Nestec S.A., under article 2(ix) of the Budapest Treaty.

ATCC refers to American Type Culture Collection, 10801 University Blvd., Manassas, Va. 20110-2209, U.S.A. CNCM refers to Collection nationale de cultures de micro-organismes, Institut Pasteur, 28, rue du Dr Roux, F-75724 Paris Cedex 15, France.

The Bifidobacterium probiotic of the invention is a live probiotic bacteria. Bacteria are considered as “live” when they are able to multiply under controlled culture conditions and form colonies or suspensions or when the microorganism metabolic activity and/or membrane integrity can be established using methods known to the person skilled in the art, such as for example flow cytometry.

Food Product

The invention further relates to a food product comprising the composition as herein described. The food product may be a cereal bar, biscuit, yoghurt, powdered beverage, or the like.

Use as a Medical Food

The invention further relates to a composition or a food product as herein described, for use as a medical food to prevent or treat a condition or disease in a subject. In some embodiments, said composition is for use as a medical food for weight management, irritable bowel syndrome, chronic enteropathy, and/or atopic dermatitis in a subject. In some embodiments, the medical food is to prevent or treat irritable bowel syndrome. In some embodiments, the medical food is to prevent or treat chronic enteropathy. In some embodiments, the medical food is to prevent or treat atopic dermatitis.

### EXAMPLES

### Example 1: Upper GIT Simulation and Short Term Colonic Incubations

The strain B. longum subsp. longum NCC 3001 (ATCC BAA-999), was used in this in vitro study. A total amount of 2.0E8 CFU was fed in the system. Pea cell wall fiber (obtained from a commercial source) and corn fiber mix (also known as Fiber1, obtained from CPW Breakfast Cereals) were used as fibers and a concentration of 22 g/L of each was fed in the system. Corn fiber mix contains 45% dietary fiber, and comprises 33.5% corn bran, 5.3% whole grain wheat flour, 5% resistant dextrin and 0.8% guar gum. Every synbiotic couple and the probiotic and prebiotics constituents alone were tested in three biological replicates.

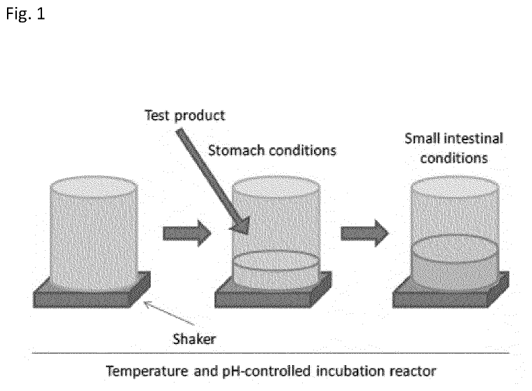

An adapted SHIME® system was used to investigate the survivability of probiotics in upper GIT (stomach and small intestine). The study was performed using a protocol that was adapted from the InfoGest consensus method and the dynamic pH profile for in vivo upper GIT study. One reactor was first subjected to stomach conditions and subsequently small intestinal conditions, mimicking both fed and fasted conditions (FIG. 1).

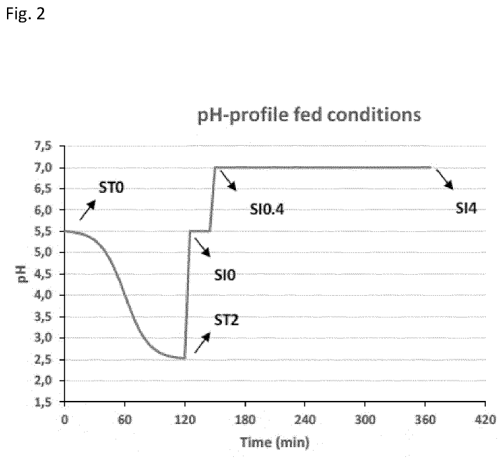

Incubation started by feeding the probiotics and fiber samples of this study with 7 g/L fructose for 2 hours at 37° C. with mixing, where pH decreased from 5.5 to 2.5 (FIG. 2). A specific gastric suspension (Pepsin, phosphatidylcholine, fructose and additional nutritional medium) was then added to simulate the stomach stage. After 5 minutes, a standardized pancreatic juice containing pancreatin from porcine pancreas and 10 mM bovine bile extract was added to simulate duodenal stage. After 20 minutes, the reactor contents were transferred to a dialysis membrane (regenerated cellulose, 3.5 kDa) which was submerged in a solution containing the same bicarbonate concentration as the content of the membrane, 4 times the volume, and at pH 7. Dialysis was performed for 3.67 hours to simulate jejunal and ileal incubation.

Short-term colonic incubations for gut microbial metabolic analysis were performed using a representative colon medium containing host-derived and diet-derived compounds and a colonic microbiota of a single healthy human adult donor. Upper GIT suspension (containing non-absorbed fraction of fructose) was administered to the colonic incubations, in three biological replicates, without additional fermentable carbohydrates, except for samples with prebiotic fibers. As control, upper GIT suspension containing only the non-absorbed fraction of fructose (no probiotic or prebiotic added) in the gastric juice was administered. Incubations were performed at 37° C. anaerobically for 48 hours at pH 6.5.

### Example 2: In Vitro Gastro Intestinal Resistance of Probiotic

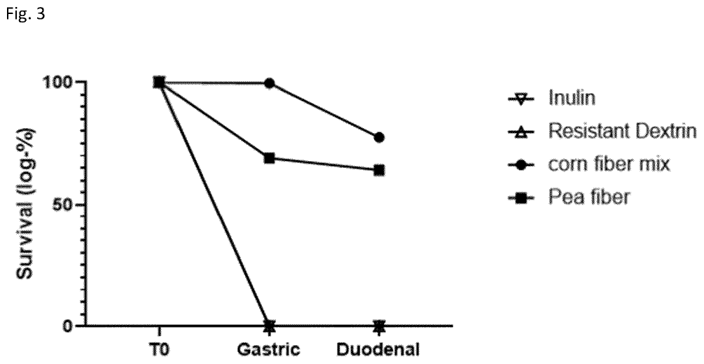

In vitro gastro intestinal resistance of probiotic upon growth on a selected set of soluble and insoluble fiber as sole carbon source (2%) was evaluated. For that purpose, the probiotic was grown in an MRS base without sugar, to which 0.05% of cysteine was added and to which 2% of the sterile fiber was added. Upon 48 hours of incubation at 37° C. in anaerobiosis, initial Colony Forming Unit (CFU) was determined and survival through a microplate simulated gastro intestinal system was evaluated. The gastric phase consisted in an incubation of 30 minutes in a solution composed of porcine pepsin (0.3%), NaCl (0.5%) adjusted at a pH of 3.5. Following that step, 1:10 of the test solution was transferred to a duodenal phase, consisting of an incubation of 60 minutes in a solution composed of porcine bile (0.49%), porcine pancreatin (0.24%) dissolved in a 0.2 M phosphate buffer at pH 7. All incubations were performed at 37° C. and CFU were evaluated after each stage. Results demonstrated that soluble fibers such as resistant dextrin or inulin did not support the survival of the probiotic in this simulated system, while non-soluble fibers such as pea fiber & corn fiber mix did support survival of the probiotic (FIG. 3).

### Example 3: Improvement of Probiotic Survival During Upper GIT Passage

Culture samples from the start and end of the stomach stage and the end of the small intestinal stage of the upper GIT incubations were collected to determine the number of colony forming units (CFU). Ten-fold dilution series were prepared in anaerobic phosphate buffered saline (PBS) and plated in petri dishes containing MRS agar medium supplemented with 0.05% cystein. Plates were incubated anaerobically for 48 hours at 37° C. Number of colonies were counted and reported below in log(CFU).

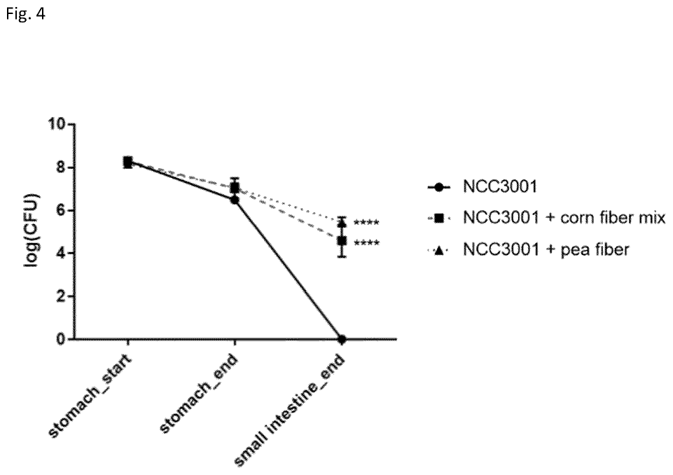

FIG. 4 shows survivability improvement of NCC 3001 through upper GIT passage when administered together with either corn fiber mix or pea fiber.

### Example 4: Improvement of Probiotic Metabolic Activity During Upper GIT Passage

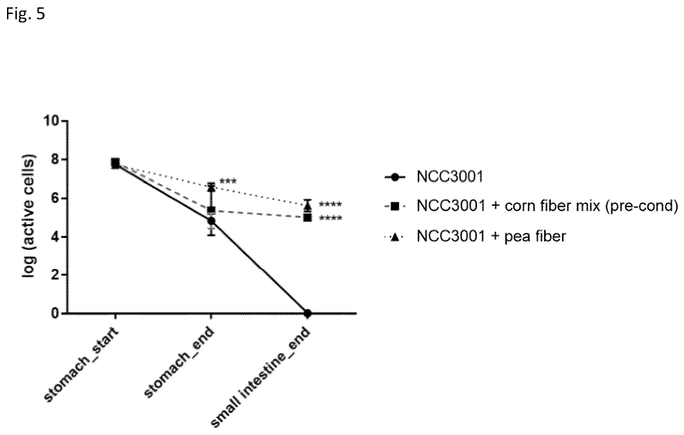

The number of metabolically active cells were also determined based on esterase activity on the marker molecule carboxyfluorescein diacetate (cFDA) and quantified by flow cytometry. Samples were stained by cFDA at appropriate dilutions and analyzed on a BDFacs verse at a high flow rate. The green-fluorescent channel was utilized to detect metabolically active cells that esterified the cFDA marker. FIG. 5 shows improvement of the number of metabolically active NCC 3001 cells when administered together with pea fiber or corn fiber mix. For the synbiotic mix of NCC 3001 with corn fiber mix, the probiotic was grown with the fiber prior to administration to the upper GIT incubations (pre-conditioned).

1. A method to improve the survival of a Bifidobacterium probiotic, comprising using Poaceae and/or a Leguminoseae plant fiber wherein said plant fiber has an insoluble fraction of between 40 to 80% (w/w) to grow the probiotic.

2. Method according to claim 1, wherein the Poaceae fiber has an insoluble fraction of between 70 to 80% (w/w).

3. Method according to claim 1, wherein the Poaceae fiber comprises corn fiber, for example a corn fiber mix comprising about 45% (w/w) corn fiber.

4. Method according to claim 1, wherein the Leguminoseae fiber has an insoluble fraction of between 40-50% (w/w) of total plant fiber.

5. Method according to claim 4, wherein the Leguminoseae fiber is pea cell wall fiber.

6. Method according to claim 1, wherein the probiotic is Bifidobacterium longum subsp. longum.

7. Method according to claim 6, wherein the probiotic is Bifidobacterium longum NCC 3001 (ATCC BAA-999).

8. Method according to claim 1, to improve metabolic activity, metabolite production, and/or bifidogenic effect of a Bifidobacterium probiotic during gastro-intestinal passage in a subject.

9. Method according to claim 1, wherein the Bifidobacterium probiotic is grown in the presence of the plant fiber, prior to administration to a subject.

10. A method of improving survival of Bifidobacterium longum wherein said method comprises growing Bifidobacterium longum in a culture medium, wherein said culture medium comprises a Poaceae and Leguminoseae plant fiber.

11. A composition comprising an effective amount of a Poaceae and/or Leguminoseae plant fiber and a Bifidobacterium probiotic, wherein said plant fiber has an insoluble fraction of between 40 to 80% (w/w).

12. A composition according to claim 11, wherein said Bifidobacterium probiotic is obtained by a process comprising the steps of
a. Fermenting the Bifidobacterium probiotic in a bacterial growth medium; and
b. Harvesting the cultured Bifidobacterium probiotic.

13. A composition according to claim 12, wherein the bacterial growth medium comprises a Poaceae and Leguminoseae plant fiber.

14. A composition according to claim 12, wherein the bacterial growth medium comprises a Poaceae plant fiber.

15. A composition according to claim 12, wherein the bacterial growth medium comprises corn fiber or corn fiber mix.

16-17. (canceled)
